(19) United States
(12) Patent Application Publication
(10) Pub. No.: US 2022/0346721 A1
(43) Pub. Date: Nov. 3, 2022

(54) Device For Supporting A Patient In Connection with A Medical Procedure

(71) Applicant: United States Government as Represented by the Department of Veterans Affairs, Washington, DC (US)

(72) Inventors: Kevin Johnson, Cincinnati, OH (US); Gregory O. Voss, Apple Valley, MN (US); Colin G. Kenow, Saint Paul, MN (US)

(21) Appl. No.: 17/731,839

(22) Filed: Apr. 28, 2022

Related U.S. Application Data
(60) Provisional application No. 63/180,967, filed on Apr. 28, 2021.

(51) Int. Cl. A61B 5/00; A61B 5/055

(52) U.S. Cl. CPC A61B 5/702; A61B 5/704; A61B 5/055

(57) ABSTRACT

An apparatus having a longitudinal axis and an upper surface for supporting an individual at least partially positioned on a bed or other support structure, such as, for example, a bed within a magnetic resonance imaging (MRI) machine, is disclosed. The individual can have hips, legs and an upper back. The apparatus comprises a bladder that is configured to be positioned beneath the hips of the individual. The bladder has a first end and an opposed second end that is spaced from the first end along the longitudinal axis. The bladder is configured to expand upon receiving air from an air supply in order to elevate the hips of the individual relative to the upper back of the individual. A valve is in fluid communication with the bladder. The valve is configured to selectively allow air from the air supply into the bladder. The apparatus can be nonferrous.

### CROSS-REFERENCE TO RELATED APPLICATIONS

This application claims priority to and the benefit of the filing date of U.S. Provisional Application No. 63/180,967, filed Apr. 28, 2021, the entirety of which is incorporated by reference herein for all purposes.

### FIELD

This disclosure relates to devices, systems, and methods for supporting a patient before, during, or after a medical procedure, such as, for example, a procedure performed using a magnetic resonance imaging machine.

### BACKGROUND

Medical procedures, such as imaging with a magnetic resonance imaging (MRI) machine, require proper positioning of a patient. As shown in FIG. 1, many patients, such as those suffering from kyphosis (e.g., hunching of the back), scoliosis, or other spinal malformations, cannot lie perfectly flat on a bed (e.g., MRI bed) or other surface on which the patient is positioned during a procedure. Accordingly, as shown in FIG. 2, conventionally, technologists have to lift the patient and position pillows underneath the patient in order to prop the patient up in a desired position. Then, the technologists often have to lift the patient from the lying position to the sitting position. Such manipulation of the patient can be difficult, and over 65% of technologists have reported some sort of back injury during their careers.

As the population ages, an increasing number of patients require MRI scans. Roughly 20% of patients require some sort of assistance in sitting up or lying down before or after an MRI scan. Accordingly, there is a need for devices, systems, and methods that assist the technologists with properly positioning the patient during performance of a medical procedure, such as an MRI procedure.

### SUMMARY

Described herein, in various aspects, is an apparatus for supporting an individual at least partially positioned on a bed, such as, for example, a bed within a magnetic resonance imaging (MRI) machine. The individual can have hips, legs and an upper back. The apparatus can have a longitudinal axis and an upper surface. The apparatus can comprise a bladder that is configured to be positioned beneath the hips of the individual. The bladder can have a first end and an opposed second end that is spaced from the first end along the longitudinal axis. The bladder can be configured to expand upon receiving air from an air supply in order to elevate the hips of the individual relative to the upper back of the individual. A valve can be in fluid communication with the bladder. The valve can be configured to selectively allow air from the air supply into the bladder. Optionally, the apparatus can be nonferrous to permit use with an MRI machine.

In one aspect, a system includes a magnetic resonance imaging (MRI) machine, the MRI machine having a bed with a top portion. An apparatus is positioned on the top portion of the bed, the apparatus having a longitudinal axis and an upper surface. The apparatus includes a bladder that is configured to be positioned beneath hips of an individual. The bladder has a first end and an opposed second end that is spaced from the first end along the longitudinal axis. The bladder is configured to expand upon receiving air from an air supply in order to elevate the hips of the individual relative to an upper back of the individual. A valve is in fluid communication with the bladder. The valve is configured to selectively allow air from the air supply into the bladder. Optionally, the apparatus can be nonferrous to permit use with an MRI machine.

In one aspect, a method of using a magnetic resonance imaging (MRI) machine is disclosed. The MRI machine comprises a head coil and a bed having a top portion. The method includes positioning an individual on an apparatus, the apparatus having a longitudinal axis and an upper surface. The apparatus includes a bladder that is configured to be positioned beneath hips of an individual. The bladder has a first end and an opposed second end that is spaced from the first end along the longitudinal axis. The bladder is configured to expand upon receiving air from an air supply in order to elevate the hips of the individual relative to an upper back of the individual. A valve is in fluid communication with the bladder. The valve is configured to selectively allow air from the air supply into the bladder. The apparatus is nonferrous. The method further includes positioning the individual so that the hips of the individual are above the bladder and inflating the bladder until a lower portion of the body is sufficiently elevated that the head of the patient is receivable within the head coil.

In one aspect, an apparatus for supporting an individual at least partially positioned on a bed is disclosed, the individual having hips, legs and an upper back. The apparatus has a longitudinal axis and an upper surface. The apparatus includes a first bladder that is configured to be positioned beneath the hips of the individual. The first bladder has a first end and an opposed second end that is spaced from the first end along the longitudinal axis and a midpoint centered between the first end and the second end. The first bladder is configured to expand upon receiving air from an air supply in order to elevate the hips of the individual relative to the upper back of the individual. The second end of the first bladder is spaced from the first end of the first bladder in a first direction along the longitudinal axis. The apparatus further includes second bladder having a first end and an opposed second end that is spaced from the first end along the longitudinal axis and a midpoint centered between the first end and the second end. The midpoint of the second bladder is axially spaced along the longitudinal axis in the first direction from the midpoint of the first bladder. The second bladder is configured to be positioned beneath the lower back of the individual. The second bladder is configured to expand upon receiving air from the air supply in order to elevate the upper back of the individual relative to the hips of the individual. A valve system is configured to selectively allow air from the air supply into one of the first or second bladders and selectively release air from the first and second bladders.

Additional advantages of the invention will be set forth in part in the description that follows, and in part will be obvious from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DESCRIPTION OF THE DRAWINGS

These and other features of the preferred embodiments of the invention will become more apparent in the detailed description in which reference is made to the appended drawings wherein:

FIG. 1 is a perspective view of a conventional MRI machine.

FIG. 2 is a perspective view of the conventional MRI machine of FIG. 1, illustrating the conventional method for positioning an individual having a spinal malformation.

FIG. 3 is a perspective view of a system comprising an MRI machine and an apparatus as disclosed herein.

FIG. 4 is a schematic side view of an apparatus in a deflated condition in accordance with embodiments disclosed herein.

FIG. 5 is a schematic view of the apparatus as in FIG. 4 with a first bladder in an inflated condition.

FIG. 6 is a schematic view of the apparatus as in FIG. 4 with a second bladder in an inflated condition.

FIG. 7 is a perspective view of an exemplary apparatus having first and second bladders as disclosed herein.

FIG. 8 is a perspective view of the apparatus of FIG. 7 with the first bladder inflated.

FIG. 9 is a perspective view of the apparatus of FIG. 7 with the second bladder inflated.

FIG. 10 is a side view of apparatus of FIG. 7 with the first bladder inflated and a user on the apparatus.

FIG. 11 is a side view of apparatus of FIG. 7 with the second bladder inflated and a user on the apparatus.

FIG. 12 is a see-through perspective view of an exemplary bladder, showing baffles in an exemplary configuration inside the bladder.

FIG. 13 is an exemplary valve system for operating the apparatus of FIG. 7.

FIG. 14 is an underside view of the apparatus of FIG. 7.

FIG. 15 is a side view of the apparatus of FIG. 7, showing a rapid deflation system.

FIG. 16 is a block diagram of an exemplary air control system as disclosed herein.

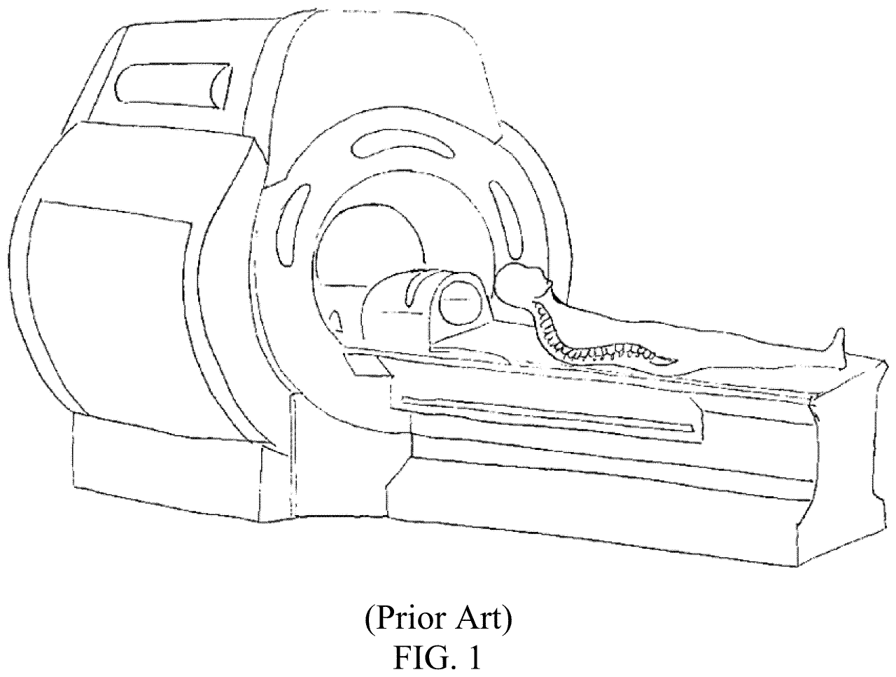

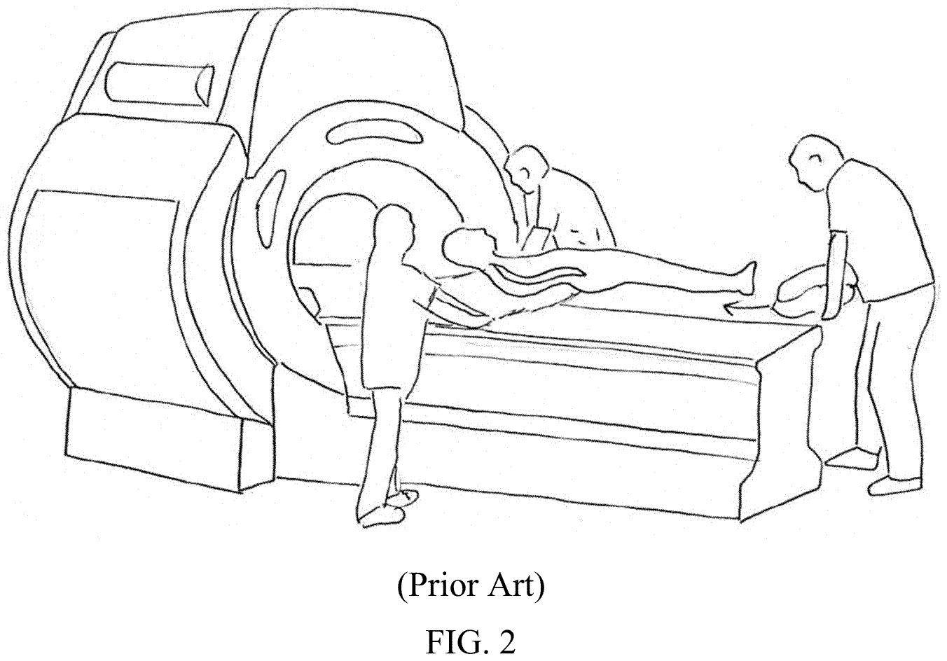

### DETAILED DESCRIPTION

The present invention now will be described more fully hereinafter with reference to the accompanying drawings, in which some, but not all embodiments of the invention are shown. Indeed, this invention may be embodied in many different forms and should not be construed as limited to the embodiments set forth herein; rather, these embodiments are provided so that this disclosure will satisfy applicable legal requirements. Like numbers refer to like elements throughout. It is to be understood that this invention is not limited to the particular methodology and protocols described, as such may vary. It is also to be understood that the terminology used herein is for the purpose of describing particular embodiments only, and is not intended to limit the scope of the present invention.

Many modifications and other embodiments of the invention set forth herein will come to mind to one skilled in the art to which the invention pertains having the benefit of the teachings presented in the foregoing description and the associated drawings. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

As used herein the singular forms “a,” “an,” and “the” include plural referents unless the context clearly dictates otherwise. For example, use of the term “a bladder” can refer to one or more of such bladders, and so forth.

All technical and scientific terms used herein have the same meaning as commonly understood to one of ordinary skill in the art to which this invention belongs unless clearly indicated otherwise.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. Optionally, in some aspects, when values are approximated by use of the antecedent “about,” it is contemplated that values within up to 15%, up to 10%, up to 5%, or up to 1% (above or below) of the particularly stated value can be included within the scope of those aspects. Similarly, in some optional aspects, when values are approximated by use of the terms “substantially” or “generally,” it is contemplated that values within up to 15%, up to 10%, up to 5%, or up to 1% (above or below) of the particular value can be included within the scope of those aspects. When used with respect to an identified property or circumstance, “substantially” or “generally” can refer to a degree of deviation that is sufficiently small so as to not measurably detract from the identified property or circumstance, and the exact degree of deviation allowable may in some cases depend on the specific context.

As used herein, the terms “optional” or “optionally” mean that the subsequently described event or circumstance may or may not occur, and that the description includes instances where said event or circumstance occurs and instances where it does not.

As used herein, the term “at least one of” is intended to be synonymous with “one or more of.” For example, “at least one of A, B and C” explicitly includes only A, only B, only C, and combinations of each.

Except when the context indicates otherwise, the word “or” as used herein can mean any one member of a particular list or can include any combination of members of that list.

It is to be understood that unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect. This holds for any possible non-express basis for interpretation, including: matters of logic with respect to arrangement of steps or operational flow; plain meaning derived from grammatical organization or punctuation; and the number or type of aspects described in the specification.

The following description supplies specific details in order to provide a thorough understanding. Nevertheless, the skilled artisan would understand that the apparatus, system, and associated methods of using the apparatus can be implemented and used without employing these specific details. Indeed, the apparatus, system, and associated methods can be placed into practice by modifying the illustrated apparatus, system, and associated methods and can be used in conjunction with any other apparatus and techniques conventionally used in the industry.

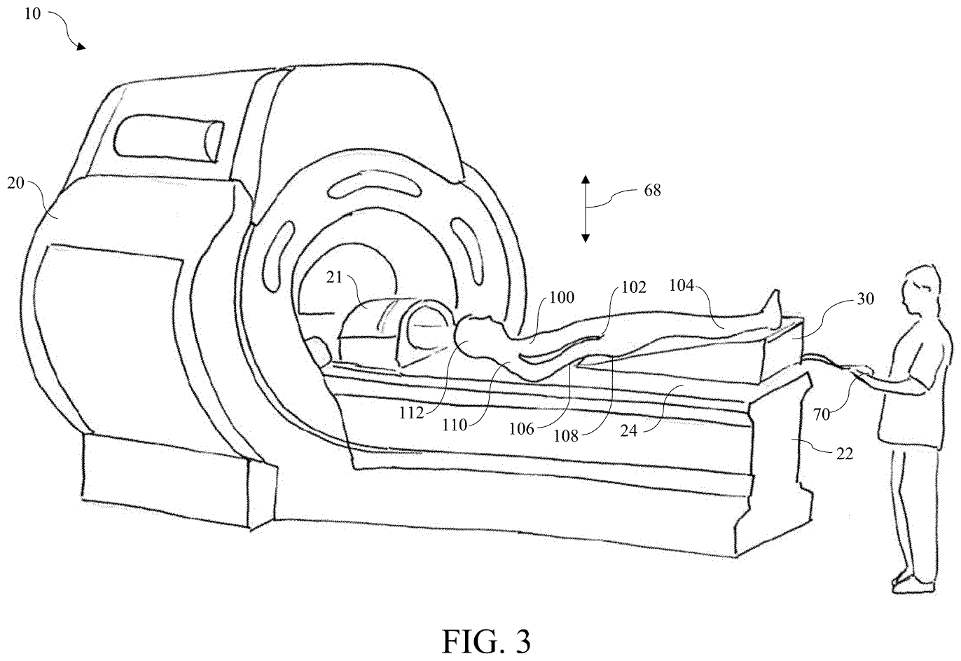

Disclosed herein, in various aspects and with reference to FIGS. 3-6 is a system 10 comprising an apparatus for performing a medical procedure (shown representatively, and without limitation, as an MRI machine 20) and an apparatus 30 for positioning an individual 100 having hips 102, legs 104, a lower back 106, a buttocks 108, an upper back 110, and a head 112. The MRI machine 20 can comprise a coil (e.g., a head coil 21) and a bed 22 having a top portion 24 upon which the apparatus 30 can be positioned. The apparatus 30 can have a longitudinal axis 32 and an upper surface 34. In use, the individual can rest on at least a portion of the upper surface 34 of the apparatus 30. Although shown representatively as an MRI machine, it is contemplated that the apparatus for performing the medical procedure can comprise any structure that is capable of supporting an individual during a medical procedure, including, for example and without limitation, an operating room table, a support platform or bed within a medical system (e.g., an imaging system), a hospital bed, or the individual's personal bed.

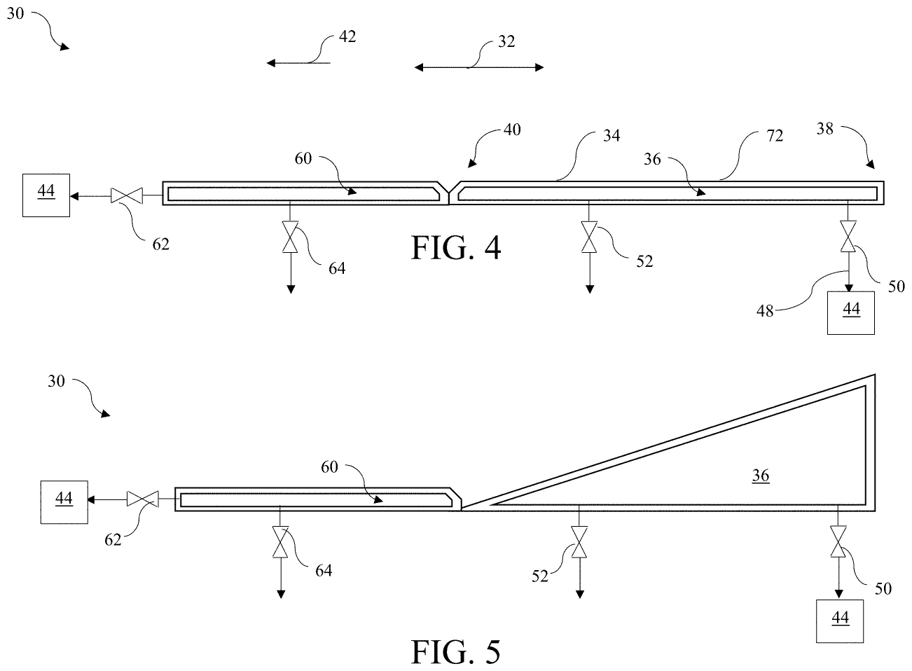

With reference to FIGS. 4-6, the apparatus 30 can comprise a first bladder 36 that can be configured to be positioned beneath the hips 102 of the individual 100. In further aspects, the first bladder 36 can be configured to be positioned beneath the hips 102 and legs 104 of the individual. In some optional aspects, the first bladder can be at least 20 inches long, at least 30 inches long, at least 40 inches long, or from about 50 inches to about 80 inches long, or about 65 inches long, or from about 25 inches long to about 40 inches long, or about 30 inches. In some optional aspects, the first bladder can have a width of at least 15 inches, or from about 15 inches to about 30 inches, or about 20 inches. The first bladder 36 can have a first end 38 and a second end 40, wherein the first end 38 is spaced from the second end 40 in a first direction 42 along the longitudinal axis 32. With the first bladder 36 at least partially positioned beneath the hips of the individual, the first bladder can be configured to expand upon receiving air from an air supply 44 in order to elevate the hips of the individual relative to the upper back of the individual.

In various aspects, the air supply 44 can comprise (or be) a pump. The pump can be in a location outside of a room in which the MRI machine 20 is located. In further aspects, the air supply can comprise (or be) a hospital/clinic air supply line. The air supply 44 can be a medical gas supply. When the apparatus 30 is used during an MRI procedure, it is contemplated that the pump, which typically comprises metal components, must either be positioned outside the room containing the MRI machine or otherwise shielded from the MRI machine. However, when the apparatus 30 is used in other applications where an MRI machine is not used, then it is contemplated that the air supply 44 can be integrated into or permanently coupled to the apparatus 30, thereby providing a self-contained and portable system for positioning and supporting an individual in the manner disclosed herein.

A first valve 50 can be in fluid communication with the first bladder 36. The first valve 50 can be configured to selectively allow air into the first bladder 36. Optionally, the first valve 50 can be a check valve or other one-way valve that allows air only into the first bladder 36 (and does not allow air to exit the first bladder). Accordingly, in some optional aspects, the air supply 44 can be a pump, and the pump can be activated to deliver air into the first bladder 36. In these aspects, it is further contemplated that the pump can stop to thereby cease inflation of the first bladder 36. In further optional aspects, the first valve 50 can be, for example, a ball valve or solenoid valve (or any suitable valve) that can move between a first state, in which air is permitted to travel from the air supply 44 to the first bladder 36, and a second state, in which air flow from the air supply to the first bladder is prevented.

The apparatus 30 can comprise a conduit 48 that is in fluid communication with the first valve 50 and is configured to communicate air from the air supply 44 to the first bladder 36.

The apparatus 30 can further comprise a second valve 52 that is in fluid communication with the first bladder 36. The second valve 52 can be configured to selectively release air from the first bladder 36 (e.g., into the ambient environment). The second valve 52 can be a solenoid valve, ball valve, or any suitable valve for selectively exhausting air from the first bladder 36. In some optional aspects, the first valve 50 and the second valve 52 can be embodied as a single spool valve. In further aspects, the first and second valves 50 can be independent valves.

The apparatus 30 can further comprise a pressure relief valve (not shown) that is in communication with the first bladder 36 and configured to relieve air from the first bladder if the pressure in the first bladder exceeds a threshold.

In some optional aspects, the first bladder 36 can be elongated (i.e., have an elongated profile) along the longitudinal axis 32 between the first end 38 and the second end 40 (e.g., the length between the first end and the second end can be greater than the width of the first bladder along a transverse axis). In some aspects, when the first bladder 36 is in an inflated condition (FIG. 5), the height of the portion of the upper surface 34 of the apparatus 30 above the first end 38 of the first bladder can be greater than the height of the portion of the upper surface of the apparatus above the second end 40 of the first bladder. In some optional aspects, in the inflated condition, at least a portion of the apparatus 30 can be wedge-shaped (e.g., having a wedge-shaped cross-section). Accordingly, in some aspects, when the first bladder 36 is in an inflated condition (FIG. 5), at least a portion of the upper surface 34 of the apparatus 30 above the first bladder 36 (e.g., an uppermost region of the wedge-shaped portion) can be elevated to at least 24 inches or at least 36 inches above at least one other portion of the upper surface 34 of the apparatus 30 (e.g., a lowermost region of the wedge-shaped portion). Thus, in some aspects, it is contemplated that the feet of the patient can be elevated up to 36 inches above the lower surfaces of her body. In further aspects, the first bladder 36 can be configured to elevate the hips of the patient from zero (horizontal) to 45 degrees, or zero to 25 degrees. In some aspects, the first bladder 36 can be configured to elevate the hips of the patient by from about 10 to about 20 inches (e.g., about 13 inches) above the plane of the table/bed upon which the apparatus 30 is positioned.

In some aspects, when the first bladder 36 is in the inflated condition (FIG. 5), the apparatus 30 can define a structured profile that is configured to be complementary to and/or supportive of a contour of at least a portion of the buttocks 108 and/or lower back 106 of the individual 100. In some optional aspects, the upper surface 34 of the apparatus 30 can define longitudinally extending ridges or projections on opposing sides of the apparatus that inhibit the individual from sliding transversely to the longitudinal axis 32 of the apparatus. In further optional aspects, it is contemplated that the upper surface 34 of the apparatus 30 can define transversely extending ridges or projections that inhibit sliding of the individual along the longitudinal axis 32 of the apparatus.

In some optional aspects, the apparatus 30 can comprise foam or other resilient, compressible material between the first bladder 36 and the upper surface 34 of the apparatus.

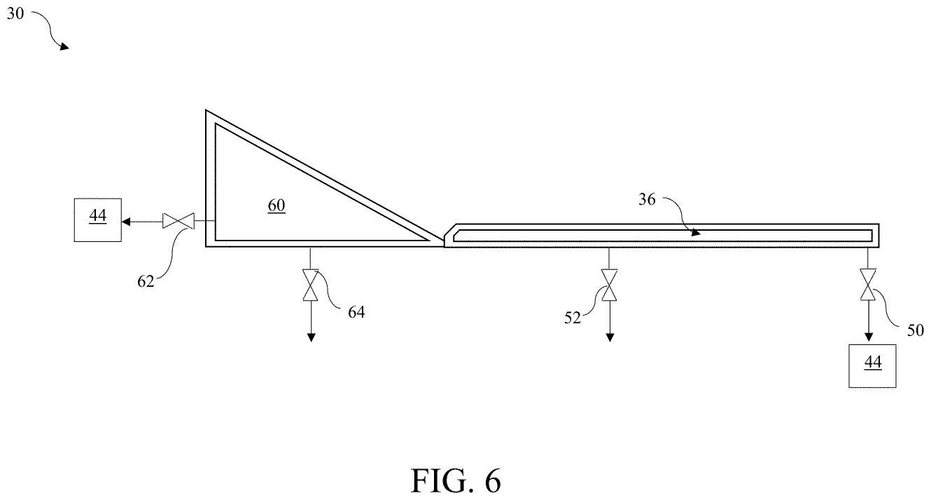

The apparatus 30 can further comprise a second bladder 60 that is spaced from the first end 38 of the first bladder 36 in the first direction 42. In use, the second bladder 60 can be configured to be positioned beneath the upper back 110 of the individual 100. In further aspects, the second bladder 60 can be configured to be positioned beneath the lower back or beneath the middle back of the individual (e.g., between the hips and the shoulder blades of the individual). The second bladder 60 can be inflated to assist the individual 100 in achieving a seated position (from a position in which the individual is laying down or otherwise positioned for a medical procedure or sleep, such as, for example with a portion of the individual within a coil of an MRI machine). Accordingly, in some aspects, when the second bladder 60 is in an inflated condition (FIG. 6), at least a portion of the upper surface 34 of the apparatus 30 above the second bladder 60 (e.g., an uppermost region of a wedge-shaped structure as described below) can be elevated to at least 12 inches or at least 24 inches or at least 36 inches above at least one other portion of the upper surface 34 of the apparatus 30 (e.g., a lowermost region of the wedge-shaped structure). The second bladder 60 can generally form a wedge shape (e.g., a three-dimensional structure having a wedge-shaped cross-section) having an angle of at least 45 degrees, or from about 45 degrees to about 75 degrees, or about 60 degrees. Thus, in some aspects, it is contemplated that the shoulders of the patient can be elevated up to 36 inches above the lower surfaces of her body. In exemplary aspects, the second bladder can be from about 10 to about 20 inches long (e.g., about 14 inches long), and from 15 to about 30 inches wide (e.g., about 20 inches wide). In the fully inflated configuration, the second bladder 60 can have a height of about 12 inches (e.g., from about 8 inches to about 16 inches).

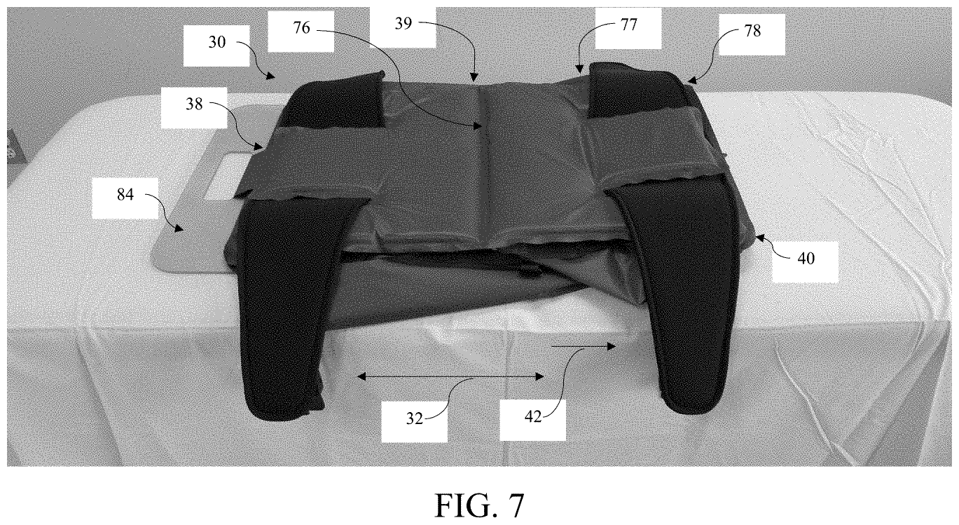

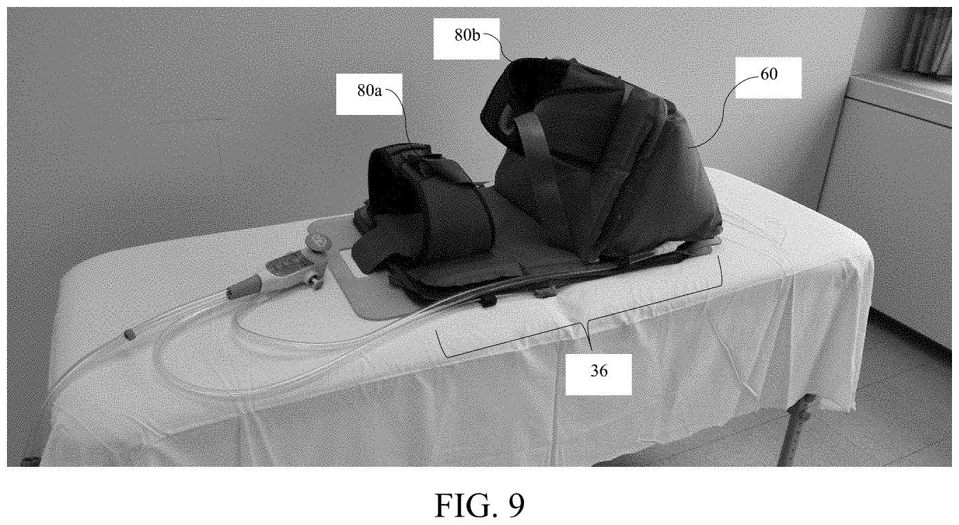

In further aspects and with reference to FIGS. 7-9, the first and second bladders 36, 60 can overlap along the longitudinal axis 32. For example, the second bladder 60 can have a first end 76 and a second end 78. In some aspects, the first end 76 of the second bladder 60 can be between the first and second ends 38,40 of the first bladder 36 along the longitudinal axis 32. In some optional aspects, the second end 78 of the second bladder 60 can be positioned at or near the second end 40 of the first bladder 36 along the longitudinal axis 32. The first end 76 of the second bladder 60 can be generally positioned below (e.g., optionally, within 2 inches, or within 4 inches of) the hips of the patient to closely align the second end 40 with the pivot point of the hips. In exemplary aspects, the first and second bladders 36, 60 can have respective midpoints 39, 77 centered between the respective first ends and second ends of the first and second bladders. The midpoint 77 of the second bladder 36 can be spaced from the midpoint of the first bladder 39 in the first direction 42 along the longitudinal axis 32.

As shown in FIG. 6, a third valve 62 can be in fluid communication with the second bladder 60. The third valve 62 can be configured to selectively allow air from the air supply 44 into the second bladder 60. A fourth valve 64 can be in fluid communication with the second bladder 60 and configured to exhaust air from the second bladder 60 (e.g., into the ambient environment). In some optional aspects, the third valve 62 can be embodied as described herein with reference to the first valve 50, and the fourth valves 64 can be embodied as described herein with reference to the second valve 52. For example, the first and third valves can be check valves or solenoid valves, ball valves, or any suitable actuatable valve. Similarly, the second and fourth valves can be solenoid valves, ball valves, or any suitable actuatable valve. The apparatus 30 can further comprise a pressure relief valve (not shown) that is in communication with the second bladder 60 and configured to relieve air from the second bladder if the pressure in the second bladder exceeds a threshold.

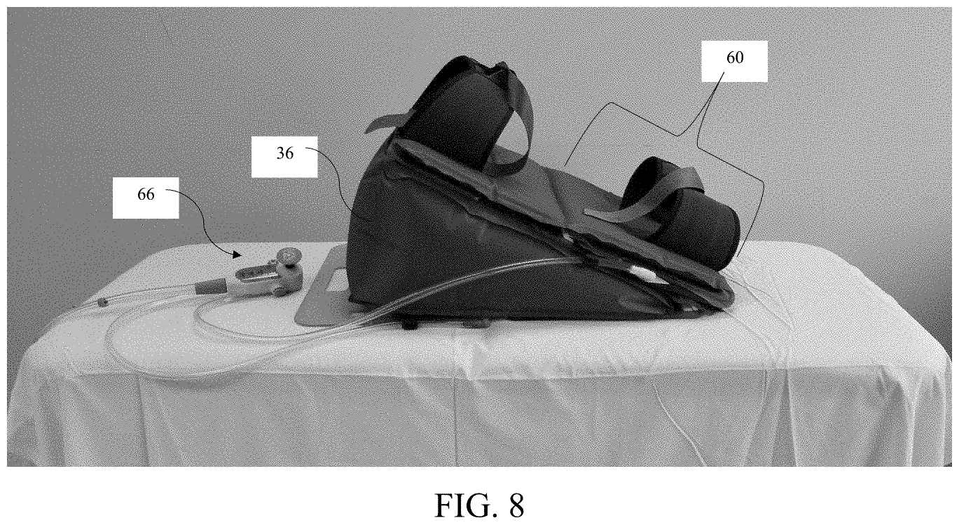

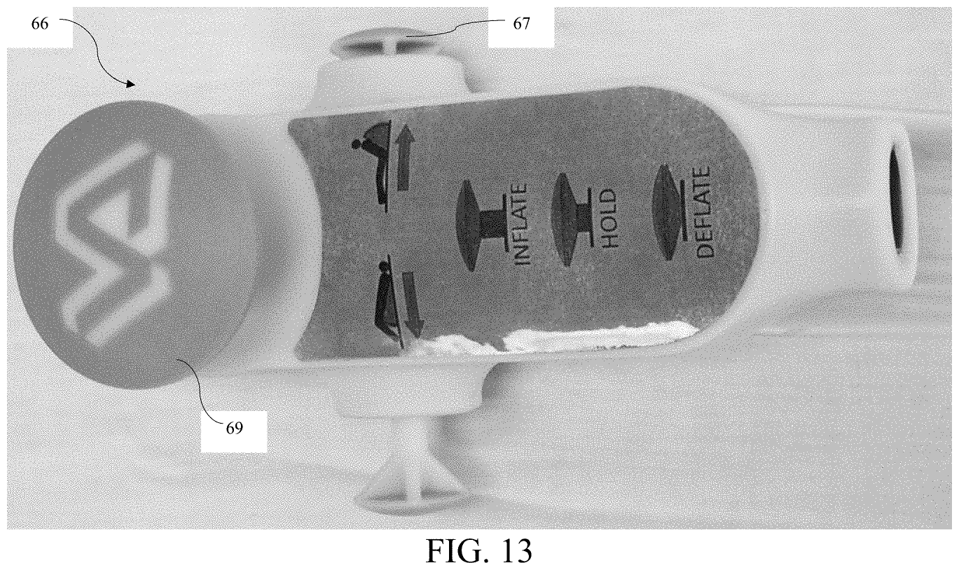

In still further aspects, and with reference to FIGS. 8 and 13, a valve system 66 can serve to inflate and deflate both the first and second bladders 36, 60. For example, in a first configuration, the valve system 66 can fluidly couple the air supply 44 with the first bladder 36 and can fluidly couple the second bladder 60 with ambient air to release any air in the second bladder. In a second configuration, the valve system 66 can fluidly couple the air supply 44 with the second bladder 60 and can fluidly couple the first bladder 36 with ambient air to release any air in the first bladder. In a third configuration, the valve system 66 can block the air supply 44 and fluidly couple both the first and second bladders 36, 60 with ambient air to deflate both bladders. Advantageously, this valve system 66 can prevent both bladders from simultaneously being inflated.

In some aspects, the valve system 66 can comprise a first valve 96 that directs flow to the first bladder or the second bladder, and a second valve 98 in line with the first valve, wherein the second valve has three states: 1) permitting flow in one direction through the first valve; 2) permitting flow in the other direction through the first valve; or 3) inhibiting flow through the first valve. The valve system 66 can have a first switch 67 that axially slides between a first position and a second position. With the first switch 67 in the first position, the valve system 66 system can provide fluid communication to the first bladder, and in the second position, the valve can provide fluid communication to the first bladder. The valve system 66 can further comprise a mode selection button 69 that selects between inflating the selected bladder, deflating the selected bladder, or holding the air in the selected bladder. In some aspects, the apparatus 30 can further comprise a pressure regulator that inhibits excessive air pressure in the bladders.

In some aspects, the valve(s) disclosed herein can selectively fluidly couple the bladders to a vacuum supply 45 (e.g., a hospital vacuum line) to more rapidly remove air from the bladders.

In some aspects, the apparatus 30 can comprise quick-disconnect fittings that permit coupling and decoupling of hoses to the bladders and valves as well as to the air supply 44 and the vacuum supply.

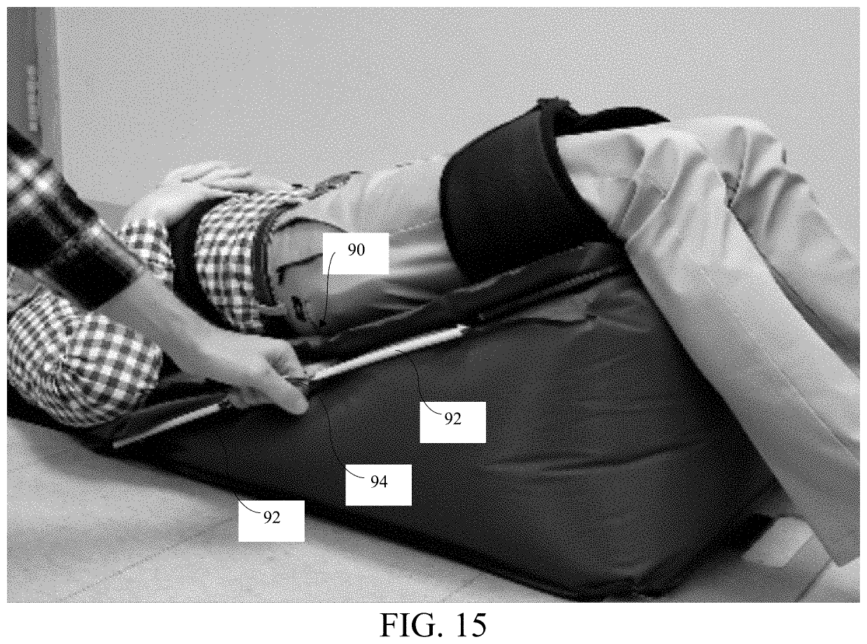

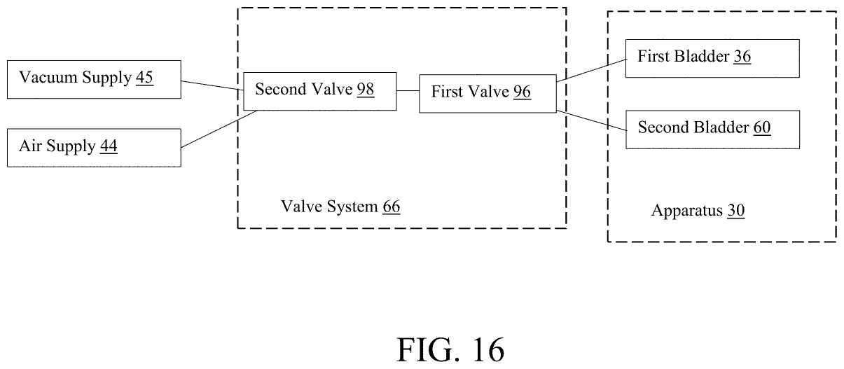

In some aspects, and with reference to FIG. 15, the apparatus 30 can comprise a rapid deflation system 90 that is configured to open up large openings in both the first and second bladders 36, 60. The rapid deflation system 90 can be used in emergency situations, such as when the individual must be removed quickly from the MRI table or dropped down to a rigid surface for medical intervention. The rapid deflation system 90 can comprise a respective clip 92 along an edge of each bladder, secured in place with plastic ties that are designed to break and give way when a quick, intentional tug is applied to the clip to expose large openings in the bladders. Both bladder clips 92 can be linked (e.g., with a cord 94, as shown) to reduce confusion as to which clip to pull in the event of an emergency. The rapid deflation system can be easily reset by re-clipping the clips over the large openings for continued use of the apparatus. Alternative rapid deflation systems can include, for example, the use of quick disconnect fittings (for example, the same quick disconnect fittings used to supply air to the bladders under normal operation), air-tight zippers, burst panels (wherein the bladder wall is torn or separated by application of external force), and/or a handle or pull cord to quickly remove the bladder from the device frame structure.

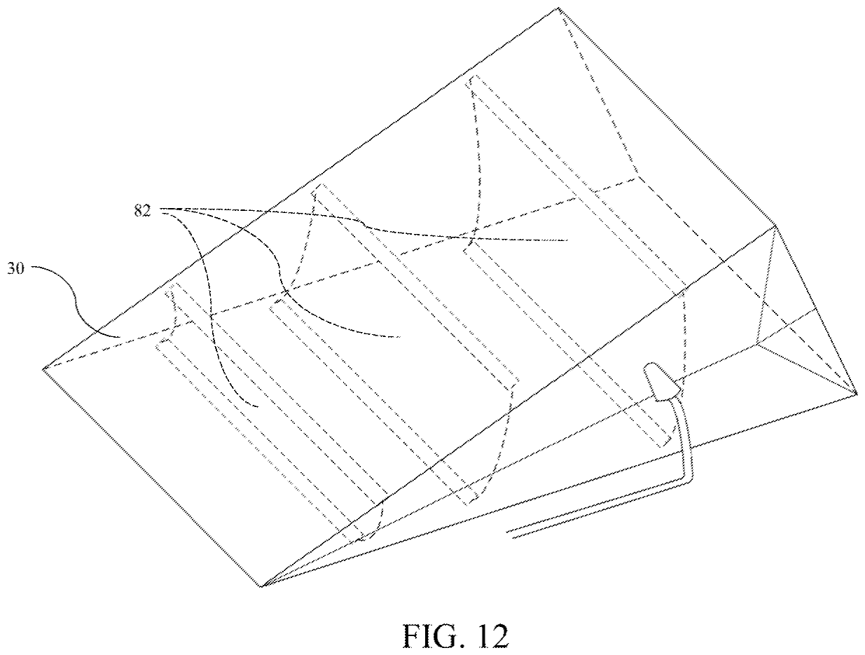

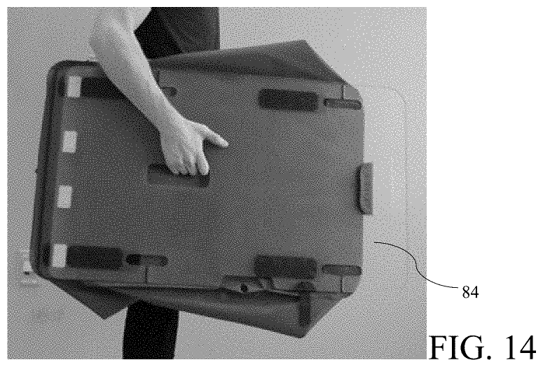

In some aspects, the apparatus 30 can comprise a housing 72 (e.g., a covering material or layer) that covers the first bladder 36. In further aspects, the housing 70 can cover both the first bladder 36 and the second bladder 60. Optionally, the housing 72 can define respective compartments within which the first and second bladders are received. Referring also to FIG. 12, in some aspects, one or both of the first and second bladders can comprise baffles 82 that control the shape of the bladders. For example, the bladders can expand until the baffles 82 reach full extension, at which point the baffles inhibit further expansion of the bladders. In exemplary aspects, the baffles 82 can comprise a plurality of baffles that are spaced along the longitudinal axis and oriented substantially transversely to the longitudinal axis. In these aspects, it is contemplated that each baffle 82 can have a different operative height to account for the variable height of the wedge-shaped structures of exemplary bladders as disclosed herein. Optionally, the apparatus 30 can comprise a hinged frame 84 (FIG. 14). The hinged frame 84 can be configured to orient the bladders, facilitate radial motion and/or expansion, provide structural support to the bladders, and/or support the individual.

In some optional aspect, it is contemplated that each of the first bladder 36 and the second bladder 60 can comprise a plurality of bladder portions that can be spaced longitudinally and/or transversely. In some aspects, the plurality of bladder portions can be simultaneously inflated/deflated. In further aspects, the plurality of bladder portions can be individually and selectively inflated/deflated. In some aspects, at least one bladder portion of the plurality of bladder portions can be spaced from at least one other bladder portion of the plurality of bladder portions that cooperatively define the first bladder 36 can be spaced along the longitudinal axis. One or more of the plurality of bladders can be individually inflated in order to, for example, raise the buttocks while lowering the legs of the patient. In further aspects, at least one bladder portion of the plurality of bladder portions can be spaced from at least one other bladder portion of the plurality of bladder portions that cooperatively define the first bladder 36 can be spaced along the a transverse axis. In this way, the plurality of bladder portions can be independently inflated to elevate one side of the patient relative to a sagittal plane to accommodate, for example, a twist in the back of the patient.

The apparatus 30 can comprise an input device 70 that can be configured to operate the apparatus. For example, the input device 70 can be configured to selectively and independently inflate and deflate the first and second bladders (optionally, selectively and independently inflate and/or deflate bladder portions within each of the first and second bladders). In some exemplary aspects, the input device can be configured to actuate each of the first, second, third, and fourth valves 50, 52, 62, 64 to selectively inflate and deflate the first and second bladders 36, 60. In embodiments in which the air supply 44 comprises a pump, the input device 70 can be configured to actuate the pump. The input device 70 can comprise, for example, one or more buttons, dials, switches, and/or knobs, a keyboard, touchscreen display, pointing device (e.g., a computer mouse or remote control), a microphone in communication with a voice-activated controller, a joystick, a scanner, tactile input devices such as gloves, and other body coverings, and/or motion sensor. In further aspects, it is contemplated that the input device 70 can comprise a computing device (e.g., smart phone, tablet, or computer) that is in communication with the pump.

Optionally, the apparatus 30 can comprise at least one strap, belt, and/or fastener that is configured to secure the individual to the apparatus. For example, with reference to FIGS. 7-11, the apparatus 30 can comprise a first strap 80a that is positioned along the longitudinal axis 32 for extending around the thighs of the individual. The apparatus 30 can further comprise a second strap 80b that is spaced from the first strap 80a along the longitudinal axis 32 and is positioned for extending around the torso (e.g., the bell or the chest) of the individual. The strap can be secured in a loop around the individual with a fastener (e.g., hook and loop, a buckle, snaps, etc.).

An entirety of the apparatus 30 can be nonferrous. For example, the apparatus 30 can comprise polymer, copper, brass, stainless steel, or titanium, or any combination thereof. In still further aspects, other nonferrous materials can be used. In this way, the apparatus 30 can be compatible for use with an MRI machine. However, in various further aspects, the apparatus 30 can be advantageously used in other situations, such as operating rooms, hospital beds, the individual's personal (home) bed, or assisted living residences. Accordingly, in further embodiments that are configured for use in situations without an MRI machine, the apparatus 30 can comprise ferrous materials.

The apparatus 30 can optionally be coupled to the bed 22 of the MRI machine 20. Optionally, a retainer can be used to keep the apparatus 30 on the bed. For example, the retainer can further comprise a channel or rail (or a pair of channels or rails) that inhibit the apparatus 30 from sliding off the bed. Optionally, the apparatus 30 can be coupled to the rails or channel (e.g., via one or more straps). The retainer can optionally be positioned on the bed or integrally formed with the bed. In further aspects, the retainer can be a cradle into which the apparatus 30 is received. Optionally, the apparatus 30 can be integrally formed with the bed 22 of the MRI machine 20. In further aspects, the apparatus 30 can be removably coupled to the bed 22 of the MRI machine 20. Optionally, the apparatus 30 can be pivotable relative to the bed 22 about a vertical axis 68, which can be perpendicular or substantially perpendicular to the longitudinal axis 32. In this way, the apparatus 30 can pivot to enable transfer of the individual onto and off of the bed. For example, the patient can be positioned on the apparatus when the apparatus is in a pivoted position in which the longitudinal axis 32 is at an acute angle with the elongation dimension of the bed 22 (measured within a horizontal plane that is perpendicular to the vertical axis 68), and the apparatus can then rotate about the vertical axis 68 to orient the patient on the bed. Still further, the apparatus can enable a technologist to account for variations in spine characteristics (e.g., a twist or a curve to the individual's left or right side).

In still further aspects, the apparatus 30 can rest on the top portion 24 of the bed 22. For example, in some optional aspects, the individual 100 can be positioned on the apparatus 30 (optionally, strapped to the apparatus), and the apparatus can be transferred from a gurney (or other wheeled bed) onto the bed 22 with the individual on the apparatus.

As described herein, the apparatus can be used to position the individual in the MRI machine 20. For example, the apparatus can be used to raise a lower portion (e.g., legs) of the body 102 of an individual having kyphosis, scoliosis, another spinal injury, or combinations thereof, in which case the individual can be considered a patient or subject. In one aspect, the individual can be positioned on the upper surface 34 of the apparatus 30, with the apparatus 30 on the top portion 24 of the bed 22. The first bladder 36 can be inflated to orient the individual. For example, the lower portion (e.g., legs 104 and/or hips 102) of the patient can be elevated until a portion of the body of the patient (e.g., the head) is flat enough to be received within a coil (e.g., head coil 21) of the MRI machine 20.

Once the individual is properly positioned at least partially within the MRI machine 20, the MRI machine can be used to generate images of the individual using conventional methods.

Once ceasing use of the MRI machine, the apparatus 30 can be used to raise the individual to a seated position. For example, the second bladder 60 can be inflated to raise the shoulders of the individual and elevate the upper back of the individual toward a seated position.

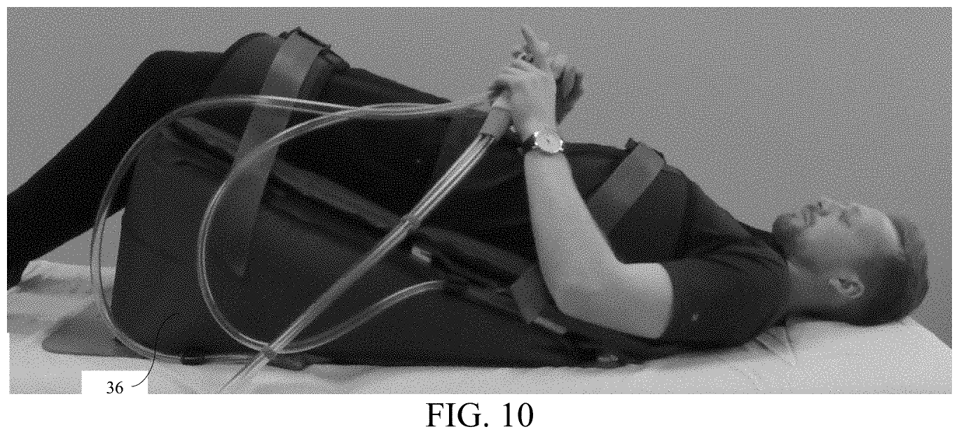

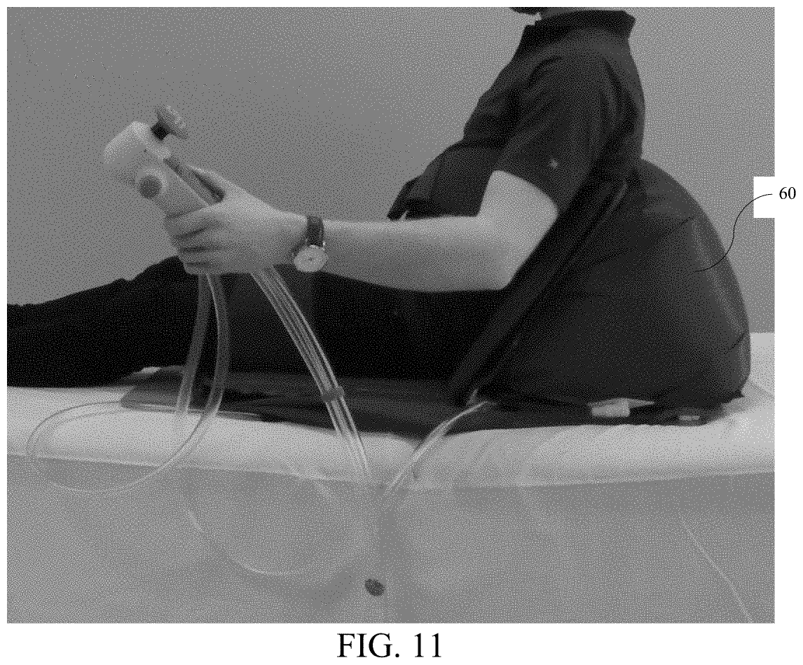

It is contemplated that the apparatus 30 can be used in an inverted orientation as compared to that shown in FIGS. 10-11. That is, the individual can lie down with her hips at the small end of the first bladder and head at the tall end. This can be used when scanning a patient's feet, legs, or hips while allowing the torso of the individual to remain elevated.

### EXEMPLARY ASPECTS

In view of the described products, systems, and methods and variations thereof, herein below are described certain more particularly described aspects of the invention. These particularly recited aspects should not however be interpreted to have any limiting effect on any different claims containing different or more general teachings described herein, or that the “particular” aspects are somehow limited in some way other than the inherent meanings of the language literally used therein.

Aspect 1: An apparatus for supporting an individual at least partially positioned within a magnetic resonance imaging (MRI) machine, wherein the individual has hips, legs and an upper back, the apparatus having a longitudinal axis and an upper surface, the apparatus comprising:

a bladder that is configured to be positioned beneath the hips of the individual, wherein the bladder has a first end and an opposed second end that is spaced from the first end along the longitudinal axis, wherein the bladder is configured to expand upon receiving air from an air supply in order to elevate the hips of the individual relative to the upper back of the individual; and
a valve in fluid communication with the bladder, wherein the valve is configured to selectively allow air from the air supply into the bladder,
wherein the apparatus is nonferrous.

Aspect 2: The apparatus of aspect 1, further comprising a conduit in fluid communication with the valve, wherein the conduit is configured to communicate air from the air supply to the bladder.

Aspect 3: The apparatus of aspect 1 or aspect 2, wherein the valve is a first valve, wherein the apparatus further comprises a second valve that is in fluid communication with the bladder and configured to be actuated to release air from the bladder.

Aspect 4: The apparatus of any one of the preceding aspects, wherein the bladder is elongated between the first end and the second end.

Aspect 5: The apparatus of any one of the preceding aspects, wherein, when the bladder is in an inflated condition, the upper surface of the apparatus above the first end of the bladder is at a greater height than the upper surface of the apparatus above the second end of the bladder.

Aspect 6: The apparatus of aspect 5, wherein the apparatus is at least partially wedge-shaped.

Aspect 7: The apparatus of any one of the preceding aspects, wherein the bladder is further configured to be positioned beneath the legs of the individual.

Aspect 8: The apparatus of any one of the preceding aspects, wherein the upper surface of the apparatus defines longitudinally extending ridges that inhibit the individual from sliding transversely to the longitudinal axis of the apparatus.

Aspect 9: The apparatus of any one of the preceding aspects, wherein the individual has a lower back and buttocks, wherein, when the bladder is in an inflated condition, the upper surface of the apparatus defines a structured profile that is configured to be complementary to a contour of at least a portion of the buttocks and the lower back of the individual.

Aspect 10: The apparatus of any one of the preceding aspects, wherein the bladder is a first bladder, wherein the first bladder has a midpoint centered between the first end and the second end, wherein the second end of the first bladder is spaced from the first end of the first bladder in a first direction along the longitudinal axis, wherein the apparatus further comprises:

a second bladder having a first end, a second end, and a midpoint centered between the first end and the second end, wherein the midpoint of the second bladder is axially spaced along the longitudinal axis in the first direction from the midpoint of the first bladder, wherein the second bladder is configured to be positioned beneath the lower back of the individual, wherein the second bladder is configured to expand upon receiving air from the air supply in order to elevate the upper back of the individual relative to the hips of the individual.

Aspect 11: The apparatus of claim 10, further comprising a rapid deflation system that is configured to, when engaged, open up large openings in both the first and second bladders.

Aspect 12: The apparatus of claim 10 or claim 11, wherein the apparatus comprises a valve system, wherein the valve system comprises the valve, wherein the valve system is configured to selectively allow air from the air supply into one of the first or second bladders and selectively release air from the first and second bladders.

Aspect 13: A system comprising:

a magnetic resonance imaging (MRI) machine, wherein the MRI machine comprises a bed having a top portion; and

an apparatus as in any one of the preceding aspects, wherein in the apparatus is positioned on a top portion of the bed.

Aspect 14: The system of aspect 13, wherein the apparatus is coupled to the bed.

Aspect 15: The system of aspect 14, wherein the apparatus is configured to rotate about a vertical axis that is perpendicular to the longitudinal axis.

Aspect 16: The system of any one of aspects 13-15, further comprising the air supply.

Aspect 17: The system of aspect 16, wherein the air supply is a pump.

Aspect 18: The system of aspect 16, wherein the air supply is a medical gas supply.

Aspect 19: The system of aspect 16, wherein the air supply is a hospital air supply line.

Aspect 20. The system of any one of aspects 13-19, further comprising a vacuum supply that is configured to be selectively fluidly coupled to the bladder.

Aspect 21: A method of using the system as in any one of aspects 13-20, wherein the MRI machine has a head coil, the method comprising:

positioning the individual on the apparatus so that the hips of the individual are above the bladder;

inflating the bladder until a lower portion of the body is sufficiently elevated that the head of the patient is receivable within the head coil.

Aspect 22: The method of aspect 21, further comprising, with the individual at least partially positioned within the MRI machine, generating, using the MRI machine, images of the individual.

Aspect 23: The method of aspect 22, the method further comprising:

ceasing use of the MRI machine; and

deflating the bladder of the apparatus.

Aspect 24: The method of aspect 23, wherein the bladder is a first bladder, wherein the first bladder has a midpoint centered between the first end and the second end, wherein the second end of the first bladder is spaced from the first end of the first bladder in a first direction along the longitudinal axis, wherein the apparatus further comprises:

a second bladder having a first end, a second end, and a midpoint centered between the first end and the second end, wherein the midpoint of the second bladder is axially spaced along the longitudinal axis in the first direction from the midpoint of the first bladder, wherein the second bladder is configured to be positioned beneath the lower back of the individual, wherein the second bladder is configured to expand upon receiving air from the air supply in order to elevate the upper back of the individual relative to the hips of the individual,

wherein the method further comprises inflating the second bladder to raise the shoulders of the individual.

Aspect 25: An apparatus for supporting an individual at least partially positioned on a bed, wherein the individual has hips, legs and an upper back, the apparatus having a longitudinal axis and an upper surface, the apparatus comprising:

a first bladder that is configured to be positioned beneath the hips of the individual, wherein the first bladder has a first end and an opposed second end that is spaced from the first end along the longitudinal axis and a midpoint centered between the first end and the second end, wherein the first bladder is configured to expand upon receiving air from an air supply in order to elevate the hips of the individual relative to the upper back of the individual, wherein the second end of the first bladder is spaced from the first end of the first bladder in a first direction along the longitudinal axis; and

a second bladder having a first end and an opposed second end that is spaced from the first end along the longitudinal axis and a midpoint centered between the first end and the second end, wherein the midpoint of the second bladder is axially spaced along the longitudinal axis in the first direction from the midpoint of the first bladder, wherein the second bladder is configured to be positioned beneath the lower back of the individual, wherein the second bladder is configured to expand upon receiving air from the air supply in order to elevate the upper back of the individual relative to the hips of the individual; and

a valve system that is configured to selectively allow air from the air supply into one of the first or second bladders and selectively release air from the first and second bladders.

Although the foregoing invention has been described in some detail by way of illustration and example for purposes of clarity of understanding, certain changes and modifications may be practiced within the scope of the appended claims.

What is claimed is:

1. An apparatus for supporting an individual at least partially positioned within a magnetic resonance imaging (MRI) machine, wherein the individual has hips, legs and an upper back, the apparatus having a longitudinal axis and an upper surface, the apparatus comprising:
a bladder that is configured to be positioned beneath the hips of the individual, wherein the bladder has a first end and an opposed second end that is spaced from the first end along the longitudinal axis, wherein the bladder is configured to expand upon receiving air from an air supply in order to elevate the hips of the individual relative to the upper back of the individual; and
a valve in fluid communication with the bladder, wherein the valve is configured to selectively allow air from the air supply into the bladder,
wherein the apparatus is nonferrous.

2. The apparatus of claim 1, further comprising a conduit in fluid communication with the valve, wherein the conduit is configured to communicate air from the air supply to the bladder.

3. The apparatus of claim 1, wherein the valve is a first valve, wherein the apparatus further comprises a second valve that is in fluid communication with the bladder and configured to be actuated to release air from the bladder.

4. The apparatus of claim 1, wherein the bladder is elongated between the first end and the second end.

5. The apparatus of claim 1, wherein, when the bladder is in an inflated condition, the upper surface of the apparatus above the first end of the bladder is at a greater height than the upper surface of the apparatus above the second end of the bladder.

6. The apparatus of claim 5, wherein the apparatus is at least partially wedge-shaped.

7. The apparatus of claim 1, wherein the bladder is further configured to be positioned beneath the legs of the individual.

8. The apparatus of claim 1, wherein the upper surface of the apparatus defines longitudinally extending ridges that inhibit the individual from sliding transversely to the longitudinal axis of the apparatus.

9. The apparatus of claim 1, wherein the individual has a lower back and buttocks, wherein, when the bladder is in an inflated condition, the upper surface of the apparatus defines a structured profile that is configured to be complementary to a contour of at least a portion of the buttocks and the lower back of the individual.

10. The apparatus of claim 1, wherein the bladder is a first bladder, wherein the first bladder has a midpoint centered between the first end and the second end, wherein the second end of the first bladder is spaced from the first end of the first bladder in a first direction along the longitudinal axis, wherein the apparatus further comprises:
a second bladder having a first end, a second end, and a midpoint centered between the first end and the second end, wherein the midpoint of the second bladder is axially spaced along the longitudinal axis in the first direction from the midpoint of the first bladder, wherein the second bladder is configured to be positioned beneath the lower back of the individual, wherein the second bladder is configured to expand upon receiving air from the air supply in order to elevate the upper back of the individual relative to the hips of the individual.

11. The apparatus of aspect 10, further comprising a rapid deflation system that is configured to, when engaged, open up large openings in both the first and second bladders

12. The apparatus of claim 10, wherein the apparatus comprises a valve system, wherein the valve system comprises the valve, wherein the valve system is configured to selectively allow air from the air supply into one of the first or second bladders and selectively release air from the first and second bladders.

13. A system comprising:
a magnetic resonance imaging (MRI) machine, wherein the MRI machine comprises a bed having a top portion; and
an apparatus positioned on the top portion of the bed, the apparatus having a longitudinal axis and an upper surface, the apparatus comprising:
a bladder that is configured to be positioned beneath hips of an individual, wherein the bladder has a first end and an opposed second end that is spaced from the first end along the longitudinal axis, wherein the bladder is configured to expand upon receiving air from an air supply in order to elevate the hips of the individual relative to an upper back of the individual; and
a valve in fluid communication with the bladder, wherein the valve is configured to selectively allow air from the air supply into the bladder,
wherein the apparatus is nonferrous.

14. The system of claim 13, wherein the apparatus is coupled to the bed.

15. The system of claim 14, wherein the apparatus is configured to rotate about a vertical axis that is perpendicular to the longitudinal axis.

16. The system of claim 13, further comprising the air supply.

17. The system of claim 16, wherein the air supply is a pump.

18. The system of claim 16, wherein the air supply is a medical gas supply.

19. The system of claim 16, wherein the air supply is a hospital air supply line.

20. The system of claim 13, further comprising a vacuum supply that is configured to be selectively fluidly coupled to the bladder.

21. A method of using a magnetic resonance imaging (MRI) machine, wherein the MRI machine comprises a head coil and a bed having a top portion, the method comprising:
positioning an individual on an apparatus, the apparatus having a longitudinal axis and an upper surface, the apparatus comprising:
a bladder that is configured to be positioned beneath hips of an individual, wherein the bladder has a first end and an opposed second end that is spaced from the first end along the longitudinal axis, wherein the bladder is configured to expand upon receiving air from an air supply in order to elevate the hips of the individual relative to an upper back of the individual; and
a valve in fluid communication with the bladder, wherein the valve is configured to selectively allow air from the air supply into the bladder,
wherein the apparatus is nonferrous,
positioning the individual so that the hips of the individual are above the bladder;
inflating the bladder until a lower portion of the body is sufficiently elevated that the head of the patient is receivable within the head coil.

22. The method of claim 21, further comprising, with the individual at least partially positioned within the MRI machine, generating, using the MRI machine, images of the individual.

23. The method of claim 22, the method further comprising:
ceasing use of the MRI machine; and
deflating the bladder of the apparatus.

24. The method of claim 21, wherein the bladder is a first bladder, wherein the first bladder has a midpoint centered between the first end and the second end, wherein the second end of the first bladder is spaced from the first end of the first bladder in a first direction along the longitudinal axis, wherein the apparatus further comprises:
a second bladder having a first end, a second end, and a midpoint centered between the first end and the second end, wherein the midpoint of the second bladder is axially spaced along the longitudinal axis in the first direction from the midpoint of the first bladder, wherein the second bladder is configured to be positioned beneath the lower back of the individual, wherein the second bladder is configured to expand upon receiving air from the air supply in order to elevate the upper back of the individual relative to the hips of the individual,
wherein the method further comprises inflating the second bladder to raise the shoulders of the individual.
